(19) United States
(12) Patent Application Publication
(10) Pub. No.: US 2005/0049507 A1
(43) Pub. Date: Mar. 3, 2005

(54) Method and measuring changes in microvascular capillary blood flow

(76) Inventors: Michael Clark, Tasmania (AU); Stephen Rattigan, Tasmania (AU); Eugene Barret, Charlottesville, VA (US); Jonathan Lindner, Charlottesville, VA (US); Sanjiv Kaul, Charlottesville, VA (US)

Correspondence Address: NIXON & VANDERHYE, PC, 1100 N GLEBE ROAD, 8TH FLOOR, ARLINGTON, VA, 22201-4714, US

(21) Appl. No.: 10/480,438

(22) PCT Filed: Jun. 11, 2002
(86) PCT No.: PCT/AU02/00753

(30) Foreign Application Priority Data
Jun. 19, 2001 (AU) PR 5769

(51) Int. Cl. A61B 8/14

(52) U.S. Cl. 600/458

(57) ABSTRACT

A method of measuring changes in microvascular capillary flow in a predetermined region in a subject comprising: (a) administering an ultrasound contrast medium to said subject such that said contrast medium reaches the microvascular capillaries in said predetermined region; (b) measuring microvascular capillary blood flow volume and/or microvascular flow index of said capillaries; (c) applying a defined signal to said subject; and, (d) measuring changes in said microvascular capillary flow.

### FIELD OF THE INVENTION

The present invention relates to the detection of impaired microvascular capillary blood flow in muscle and in particular provides methods of diagnosis and treatment of diabetes and related pathological states where insulin resistance of muscle occurs.

### BACKGROUND OF THE INVENTION

A number of laboratories have now reported an effect of insulin to increase total blood flow to muscle and that this effect is impaired in states of insulin resistance (1-3). However, the role of the increase in total blood flow mediated by insulin is controversial. Several research groups claim that insulin-mediated changes in total blood flow relate poorly to muscle glucose uptake under a number of circumstances, including insulin dose and time course (4-6). In addition there have been studies where total flow changes persist when glucose uptake is inhibited (7,8). Also, most vasodilators that augment total blood flow to the limbs do not enhance insulin action nor do they overcome insulin resistance (9, 10).

The applicants have shown that hormone and nutrient access by regulating flow between nutritive and non-nutritive flow routes in muscle is a central process in controlling both muscle metabolism and function. It has also been shown that restriction of insulin and glucose access by pharmacologically manipulating flow to be predominantly non-nutritive, creating a state of insulin resistance. The applicants then searched for a method that could detect changes in the proportion of nutritive (capillary) to non-nutritive blood flow in this tissue that might have application in vivo and ultimately to humans. Marker enzymes located in one or other of the two vascular networks (nutritive or non-nutritive) were to provide the key. Thus a first method was developed which involved 1-methylxanthine (1-MX) (20), as an exogenous substrate for xanthine oxidase, an enzyme shown by others to reside predominantly in capillary (nutritive) endothelial cells (21). 1-MX was infused intra-arterially and its metabolite 1-methyl urate measured in venous blood by HPLC; the extent of conversion a reflection of capillary exposure. Characterization under a number of conditions revealed that 1-MX metabolism was indeed directly proportional to nutritive, or capillary flow, which in the constant-flow perfused hindlimb system could be altered by applying various vasoconstrictors or by simulating exercise. The 1-MX method was tested in vivo using the hyperinsulinaemic euglycaemic clamp in rats and it was shown for the first time that insulin acted directly to recruit capillary flow in muscle. Further work showed that pharmacological manipulation to decrease the proportion of nutritive blood flow by an infused vasoconstrictor, created a state of insulin resistance. These latter findings directly linked blood pressure through blood redistribution to insulin resistance in vivo—a situation that is evident from a number of epidemiological studies of human populations. In addition, a close link between capillary recruitment and muscle glucose uptake began to emerge from these and previous 1-MX studies.

A second method was devised using the latest technologies in ultrasound.

The ultrasound method relies on the increased echogenicity of microbubbles which are continuously infused intravenously during data acquisition. The acoustic signal that is generated from the microbubbles when exposed to ultrasound produces tissue opacification which is proportional to the number of microbubbles within the ultrasound beam. Using harmonic pulsing methods essentially all microbubbles within the ultrasound beam are destroyed in response to a single pulse of high-energy ultrasound and an image is obtained. In the time interval between subsequent pulsing episodes, microbubbles flowing into the tissue are replenished within the beam and affect the intensity of the signal from the next high energy pulse. Repeating this process with pulse delays between 50 msec and 20 sec, the beam will be fully replenished and further increases in the time between each pulsing interval will not produce a change to tissue opacification. The rate of microbubble reappearance within the ultrasound beam provides an indication of capillary velocity and the degree of tissue opacification provides a measurement of capillary blood volume (CBV). Images are background-subtracted from images from a pulsing interval of 1000 ms which represents the replenishment of arteries and arterioles thus providing a measurement of capillary flow.

The plateau tissue opacification (measured as videointensity) is the determination of capillary blood volume. Using this approach, changes in capillary blood volume in response to insulin and exercise were assessed in the skeletal muscle of the rat hindlimb in vivo and compared to data obtained using 1-MX metabolism. Compared to baseline values, saline-infusion resulted in little change in capillary blood volume whereas marked increases in capillary blood volume occurred during euglycemic insulin clamp (3 mU/min/kg). Preliminary studies demonstrated that Contrast Enhanced Ultrasound (CEU) data correlates well with 1-MX metabolism data, and that capillary blood volume increases 2-3 fold during these physiologic doses of insulin. A particular advantage of the ultrasound method is that it is relatively non-invasive and is suitable for human use thereby opening up possibilities for its use in diagnosis and the monitoring of outcomes from therapeutic interventions.

Therefore, from these studies a routine diagnostic test emerges where impaired response to a signal, for example insulin, in terms of capillary recruitment can be made. This would take the form of measuring the response to a number of possible stimuli that would be expected to lead to an increased signal reflecting capillary recruitment. For insulin the stimuli may include: the response to a standard meal; insulin infusion; insulin injection; insulin inhalation; oral insulin releasing drugs; oral insulin enhancing drugs; or a combination of the aforementioned; or exercise.

Patients where the techniques and methods of the invention outlined above would be applicable include those with type 2 diabetes, type 1 diabetes, hypertension, obesity, a combination of the aforementioned and critical care patients.

### SUMMARY OF THE INVENTION

In one aspect the invention provides a method of measuring changes in microvascular capillary flow in a predetermined region in a subject comprising:
a) administering an ultrasound contrast medium to said subject such that said contrast medium reaches the microvascular capillaries in said predetermined region;.
b) measuring microvascular capillary blood flow volume and/or microvascular flow velocity index of said capillaries;
c) applying a defined signal to said subject;
d) measuring changes to said microvascular capillary flow wherein the measurement may be made by ultrasound imaging or other methods.

The ultrasound contrast medium may be microbubbles and is most preferably albumin microbubbles, although other contrast media may be used including microbubbles of similar size (3-4 micrometres diameter) and echo characteristics. e.g. phospholipid microbubbles.

The ultrasound image may be obtained by using contrast enhanced ultrasound.

The defined signal can be any thing potentially or actually affecting microvascular capillary flow; including insulin applied to said capillaries, a drug intended to increase or decrease microvascular capillary flow or a defined level of exercise applied to a muscle area associated with said capillaries.

In another aspect the invention provides a diagnostic method to differentiate normal from abnormal responses in microvascular capillaries to the application of a defined signal comprising the use of the above method to measure microvascular capillary flow in a predetermined region in a subject in response to said defined signal when compared to the flow in a control microvascular capillary region.

In another aspect the invention provides a diagnostic method incorporating the direct measurement of change in microvascular capillary flow at the source using the measurement methods as previously set out.

In another aspect the invention provides a method for determining changes in nutritive capillary recruitment using the measurement methods as previously set out.

In another aspect the invention provides a method of diagnosis of any one or a combination of conditions including Type 1 &2 diabetes, hypertension, obesity and critical care patients comprising:
a) administering an ultrasound contrast medium to a subject in need of diagnosis such that said contrast medium reaches said microcapillaries;
b) measuring microvascular capillary blood flow volume and/or microvascular flow velocity index of said capillaries;
c) applying a defined signal to said subject;
d) measuring changes to said microvascular capillary flow wherein the measurement is made by ultrasound imaging;
e) repeating steps (a) to (d) in a control subject and/or comparing known control results, such that any variation of results from the control indicates the existence or predisposition of said subject to said condition.

The defined signal may be insulin.

### DETAILED DESCRIPTION OF THE INVENTION

In order that the nature of the present invention may be more clearly understood details are given in the following particularly preferred embodiments relating to the equipment, materials and how the techniques of the invention are administered and data analyzed. The embodiments are not to be taken as limiting the scope of the invention.

Equipment

Ultrasound equipment can be Sonos 5500, Hewlett-Packard (Andover, Mass.) or equivalent. For example an HDI-5000 by ATL Ultrasound was also found to be suitable.

Microbubbles

Albumin microbubbles containing a mixture of air and perfluorocarbon gas (Optison®, Mallinckrodt Medical, Inc. St Louis, Mo.).

Procedure

This is as applied to the human forearm. Details on the forearm procedure can be found in published work which is herein incorporated by reference (27-29) and details for the use of contrast enhanced ultrasound/microbubbles in skeletal muscle are based on its use to study myocardial blood flow (30,31).

a) In response to a defined dose of insulin infused intra-arterially.

Typically, in response to low doses of insulin 0.01 to 0.05 mU/min/kg infused locally into the brachial artery, plasma insulin rises by 70-350 pM in blood perfusing forearm muscle with little or no effect on the systemic insulin, glucose, FFA, catecholamines or amino acid concentrations (29). As a result, the isolated effect of local insulin on total blood flow into the arm and glucose balance across the arm can be measured. In addition, capillary recruitment in the forearm flexor muscle can be measured using CEU. Total forearm blood flow is measured on each subject by two techniques: capacitance plethysmography and brachial artery ultrasound. For the Doppler flow measurements, an ultrasound system (Sonos 5500, Hewlett-Packard, Andover, Mass.) with a linear-array transducer is used with a transmit frequency of 7.5 MHz to allow 2-D imaging of the brachial artery in the long axis. Brachial artery diameter is measured 2 cm proximal to the tip of the arterial catheter at peak systole using on-line video calipers. A pulsed-wave Doppler sample blood volume is placed at the same location in the center of the vessel and the mean brachial artery blood velocity measured using on-line angle correction and analysis software. Brachial artery blood flow is calculated from 2-D Doppler ultrasound data using the equation: Q=vπ·(d/2)²

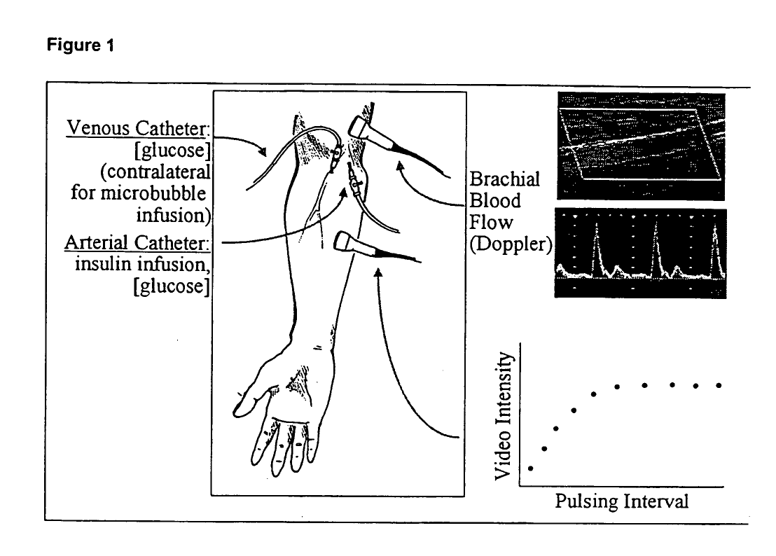

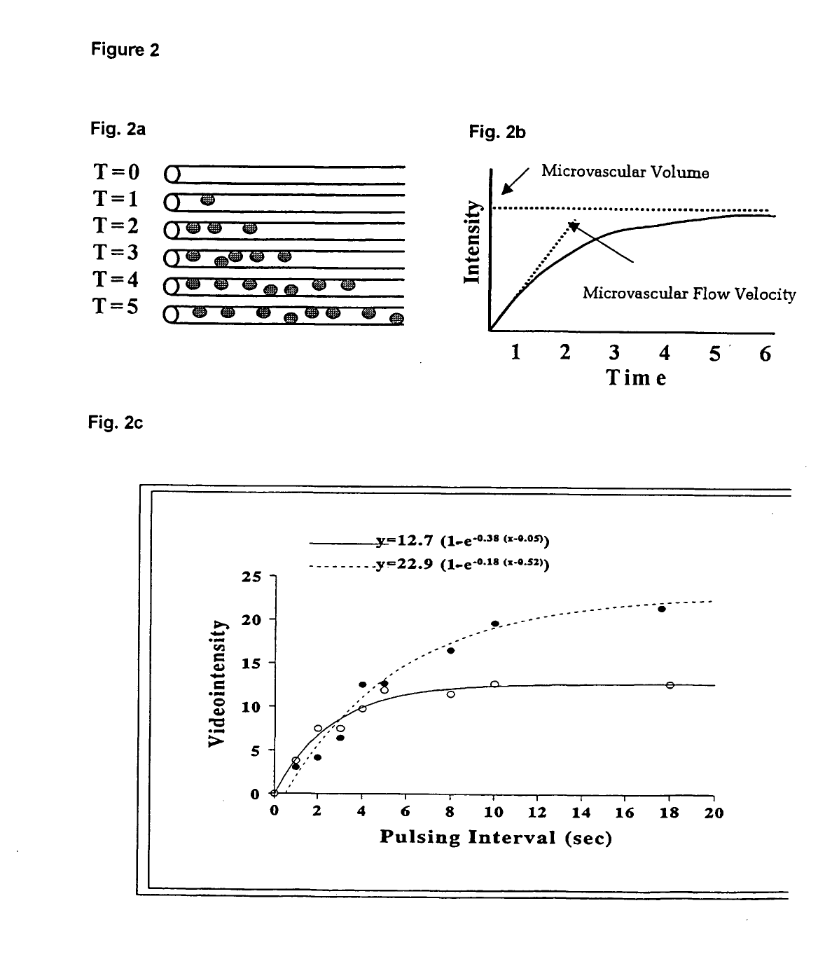

To measure capillary recruitment with CEU, a suspension of albumin microbubbles is infused intravenously in the contra-lateral arm while 2D imaging of the deep flexor muscles of the test forearm is performed (FIG. 1). FIG. 1 illustrates the experimental setup for measuring total forearm flow using Doppler ultrasound and forearm capillary recruitment using CEU as well as making glucose balance measurements across the forearm. Measurement is made in a trans-axial plane 5 cm distal to be antecubital fossa, using an ultrasound system (Sonos 5500) capable of harmonic imaging. Intermittent imaging is performed with ultrasound transmitted at 1.8 MHz and received at 3.6 MHz. Once the systemic microbubble concentration reaches steady-state (1-1.5 min), intermittent imaging is begun, at pulse intervals ranging from 1 to 15 seconds, thus allowing progressively greater replenishment of the ultrasound beam elevation between destructive pulses. Three images are acquired at each pulse interval. Additional images are acquired with the same beam characteristics at a 30 Hz sampling rate, at which there is replenishment of microbubbles only in vessels with very rapid flow, and these were used as background images. Data are recorded digitally and analyzed using custom-designed software described elsewhere (32). Averaged background frames (acquired at a 30 Hz frame rate) are digitally subtracted from the averaged frames acquired at each pulsing interval. Mean video intensity in the region of interest is measured from the background-subtracted images. Pulsing interval vs. video intensity plots are generated and fitted to an exponential function: y=A(1−e^{-βt}). Where y is the video intensity at a pulsing interval t, A is the plateau video intensity representing microvascular blood volume, and β is the rate constant reflecting the rate of rise of video intensity (and mean microbubble velocity, or microvascular flow velocity) (32,33) (FIG. 2). FIG. 2 illustrates in more detail how the microvascular blood volume or capillary volume and microvascular flow velocity are determined using CEU. FIG. 2a illustrates the successive filling of a capillary over time after all microbubbles in the capillary have been lysed by a high energy harmonic ultrasound pulse. As the delay time prior to signal detection increases (T 0 through T5) the number of microbubbles and hence the videointensity increases. FIG. 2b plots this data for typical signals collected over forearm muscle. The tangent to the upward sloping hyperbolic function is a measure of the rate of microvessel filling (MVFV) while the asymptote that intercepts the y-axis is a measure of the maximal signal seen when the vessels are filled and is determined by the microvascular volume (MVV) i.e. capillary volume. In order to derive values for the MVFV and MVV, the time versus video intensity plots are fitted to the function: Y=A(1−e^{βt}), where Y is the video intensity at time t, A is the plateau intensity which represents MVV, and β is the time constant of rise and reflects velocity. FIG. 2c is a typical experiment done before (open circles) and after (filled circles) infusing insulin (3 mU/min/kg) to an anesthetized rat. The plateau videointensity (A) is clearly higher, with no change in the rate of microvascular filling (β).

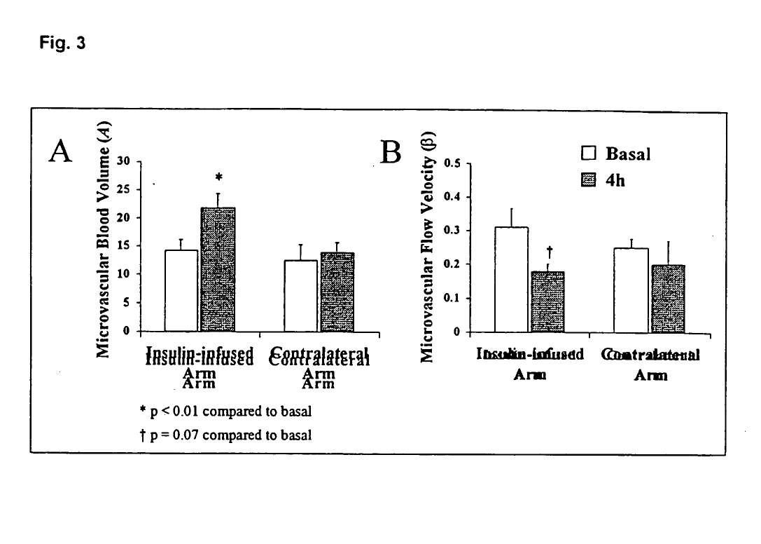

Typical data for normal healthy subjects are shown in FIG. 3. Shown here are the changes in microvascular blood volume (FIG. 3a) and microvascular flow velocity or β (FIG. 3b) in the insulin infused and contralateral human forearm of 7 subjects at basal and at 4 hrs of a 0.05 mU/min/kg brachial artery insulin infusion. * indicates p values significantly different from basal 0.05.

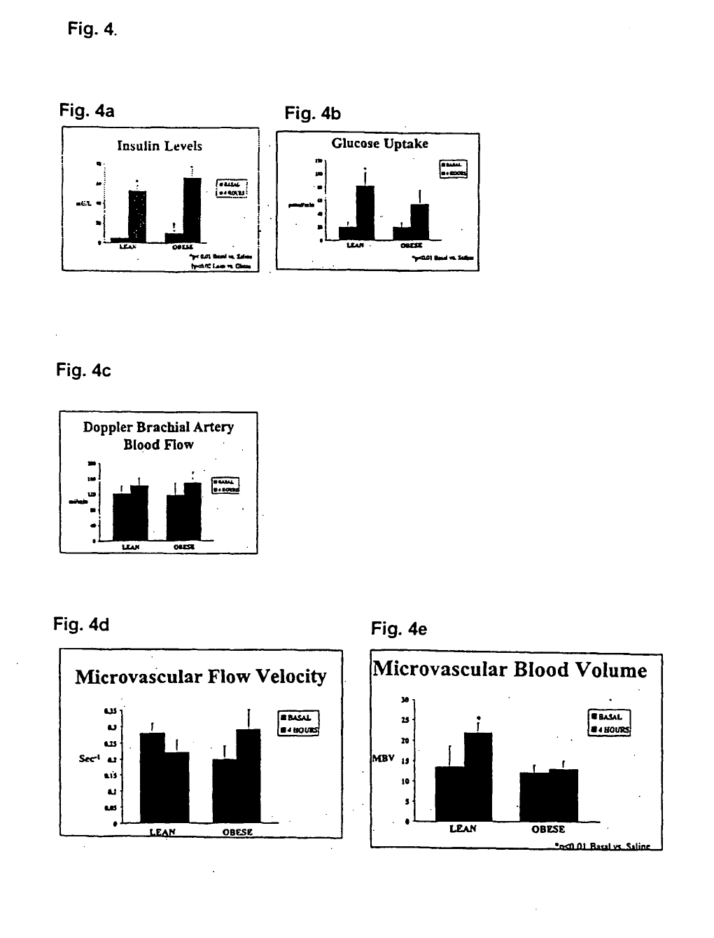

Typical data for lean normal and obese subjects are compared in FIG. 4. In healthy humans high insulin concentrations increase muscle blood flow (measured as total limb flow). in states of insulin resistance, such as Type 2 diabetes, obesity and hypertension, this vasoactive action of insulin is reported to be diminished. Obesity is associated with resistance to insulin-mediated glucose disposal and diminished skeletal muscle capillary density by histology. Whether physiologic insulin concentrations enhance muscle glucose metabolism by effects on muscle vasculature, and whether insulin's vascular actions are blunted by insulin resistant states, such as obesity, is controversial. All previous studies of the vascular effects of insulin have relied on measurements of total blood flow. Contrast˜enhanced ultrasound (CEU) combines ultrasound imaging with infusion of albumin-coated microbubbles and allows measurement of capillary blood volume in vivo in humans. Combining CEU with an established forearm perfusion method, we compared the effects of physiologic levels of insulin on total blood flow, microvascular blood volume and flow velocity, and glucose uptake in lean and obese subjects.

Methods: Subjects:—11 Lean Healthy adults (23.8±0.8 kg/m² BMI, 29±2 years old) and 6 Obese healthy adults (34.6±1.8 kg/m² BMI, 39±3 years old). CEU estimation of microvascular blood volume (MBV) and flow velocity (MBV) in forearm skeletal muscle measured by video intensity. Catheter placement in brachial artery in one forearm for insulin infusion and deep retrograde antecubital vein for blood sampling. Net glucose metabolism is measured over time by arterio-venous concentration difference multiplied by total blood flow. Protocol: Insulin was infused intra-arterially at a rate of 0.05 mU/min/kg for 4 hours. Brachial artery and bilateral deep venous blood samples were obtained at baseline and post-insulin infusion for measurements of glucose, lactate, oxygen saturation and insulin levels. Doppler U/S measured total blood flow. MBV and MBF were assessed using CEU.

In lean individuals, physiologic hyperinsulinemia increases microvascular blood volume suggesting evidence of capillary recruitment in forearm skeletal muscle. This action of insulin can occur without significant changes in total blood flow. In insulin-resistant obese individuals, this action of insulin is absent.

b) In response to a standard meal which in healthy individuals raises the insulin levels 34 fold above the pre-meal level. Leg glucose uptake increases at least four fold by 30 minutes after the meal (34), which consists of pizza (8.5 kcal/kg body weight, containing 75 g as carbohydrate, protein 37 g and fat 17 g). Other details are as above in a).

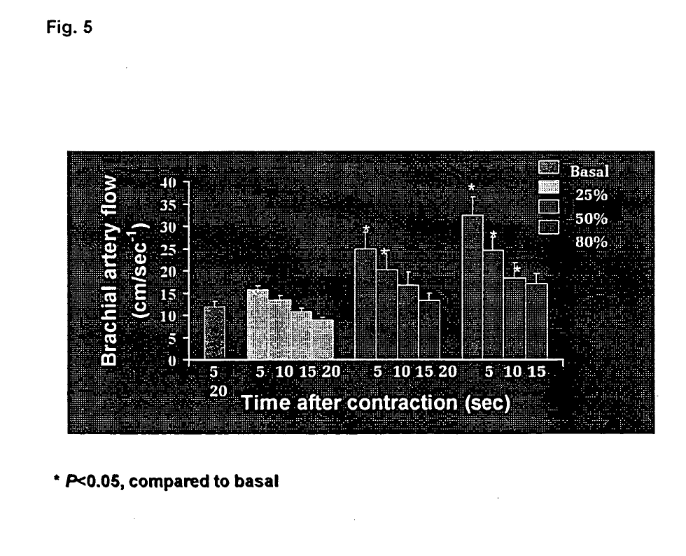

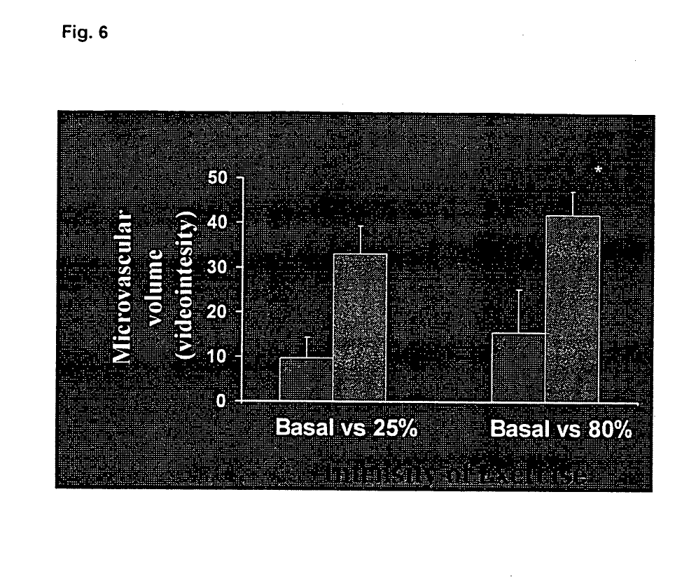

c) In response to a defined amount of exercise. The forearm is exercised by contracting to 75% maximum using a digital grip strength dynamometer (Takei Scientific Co) 12 times each minute for 2 minutes, then 3 times each minute for the next 20 min. During the last 10 min, quadruplicate blood samples, total blood flow and CEU measurements are repeated as at basal in both forearms. This leads to a 3-fold increase in forearm total blood flow. Other details are as above in a). Typical data for a lean normal subject engaged in exercising the forearm are shown in FIGS. 5 and 6. Referring to FIG. 5, in healthy humans exercising the forearm increases muscle blood flow, measured as total brachial artery flow. The increase in flow is proportional to the level of exertion, being greatest at 80% of maximum. Capillary recruitment is evidenced by increased CEU signal shown in FIG. 6. Referring to FIG. 6, in the same healthy humans as in FIG. 5, microvascular blood volume measured by CEU, increases maximally even at the lowest level of exertion that was tested (25%). These data confirm that CEU detects changes in capillary recruitment consistent with the known physiology of exercise.

Comparing FIGS. 3 and 6 indicate that exercise and insulin have similar effects to increase capillary recruitment as measured by CEU, although the exercise effect is the greater.

d) In response to a drug intended and designed to act specifically by increasing skeletal muscle capillary blood flow. These will be taken by the normal oral route and intended to achieve the afore-mentioned effect on muscle capillary blood flow acutely (within 20-60 minutes).

A number of responses were recorded on normal healthy male and female individuals at various ages. This provided a reference set of data which reflects a normal response to the defined dose of insulin and the defined exercise routine for each sex at each age. The male response could reasonably be expected to be larger than that of females at each age. However, the response would be expected to decline with age after about forty. Individuals considered to have muscle insulin resistance showed reduced responses to the defined dose of insulin and the standard meal without change to the response to exercise. This group would include type 1 and 2 diabetics, hypertensives, and the obese (>30 Body Mass Index). Critical care patients would show markedly decreased responses to all three, defined insulin dose, standard meal, and defined exercise regimen.

The efficacy of drugs intended to ameliorate skeletal muscle insulin resistance by specifically acting to increase capillary recruitment or by enhancing insulin's action to do this, will increase the response determined by CEU to the defined insulin dose, the standard meal, but not to the exercise regimen. Little or no effect is expected by these drugs in normal healthy individuals.

Any discussion of references, documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application. Any such references are herein incorporated in the application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Baron A D, Laakso M, Brechtel G, Edelman S V: Mechanism of insulin resistance in insulin dependent diabetes mellitus: a major role for reduced skeletal muscle blood flow. J. Clin. Endocrinol. Metab. 73:637-643,1991
Baron A D, Steinberg H, Brechtel G, Johnson A: Skeletal muscle blood flow independently modulates insulin-mediated glucose uptake. Am J Physiol 266:E248-E253, 1994
Baron A D: Insulin and the vasculature-old actors, new roles. J Investig. Med. 44:406-412, 1996
Utriainen J, Malmstrom R, Makimattila S, Yki-Jarvinen H: Methodological aspects, dose-response characteristics and causes of interindividual variation in insulin stimulation of limb blood flow in normal subjects. Diabetologia 38:555-564, 1995
Laine H, Yki-Jarvinen H, Kirvela O, Tolvanen J, Raitakari M, Solin O, Haaparanta M, Knnuti J, Nuutila P: Insulin resistance of glucose uptake in skeletal muscle cannot be ameliorated by enhancing endo
Yki-Jarvinen H, Utriainen T: Insulin-induced vasodilatation: physiology or pharmacology? Diabetologia 41:369-379,1998
Scherrer U, Randin D, Vollenweider P, Vollenweider L, Nicod, P: Nitric oxide release accounts for insulin's vascular effects in humans. J. Clin. Invest. 94:2511-2515, 1994
Vollenweider L, Tappy L, Owlya R, Jequier E, Nicod P, Scherrer U: Insulin-induced sympathetic activation and vasodilation in skeletal muscle. Diabetes 44:641-645, 1995
Natali A, Bonadonna R, Santoro D, Galvan A, Baldi S, Frascerra S, Palombo C, Ghione S, Ferrannini E. Insulin resistance and vasodilation in essential hypertension. J Clin Invest 94:1570-1576,1994
Sarabi M, Lind L, Millgard J, Hanni A, Hagg A, Berne C, Lithell H: Local vasodilatation with metacholine, but not with nitroprusside, increases forearm glucose uptake. Physiol Res 48:291-295, 1999
Clark M G, Colqhuoun E Q, Rattigan, S, Dora K A, Eldershaw T P D, Hall J L & Ye J. Vascular and endocrine control of muscle metabolism. Am. J. Physiol. 268: E797-E812, 1995.
Clark M G, Rattigan S, Dora K A, Newman J M B, Steen J T, Miller K A & Vincent M A. Vascular and metabolic regulation of muscle. In: Physiology, Stress and Malnutrition (Kinney J M & Tucker H, eds). Lippincott-Raven: New York, pp. 325-346,1997.
Baron A D & Clark M G. Role of blood flow in regulating glucose metabolism. Ann. Rev. Nutr. 17: 487-499, 1997.
Clark M G, Rattigan S, Dora K A, Newman J M B & Vincent M A. Interaction between blood flow, metabolism and exercise. In: Biochemistry of Exercise X (Hargreaves M, ed.). Human Kinetics: Champaign, pp. 3546, 1998.
Clark M G, Newman J M B & Clark A D H. Microvascular regulation of muscle metabolism. In: Nutrition in the ICU: Curr. Opin. Nutr. Metab. Care 1: 205-210, 1998.
Clark M G, Rattigan S, Clerk L H, Vincent M A, Clark A D H, Youd J M & Newman J M B. Nutritive and non-nutritive blood flow: rest and exercise. Acta Physiol. Scand. 168: 519-530, 2000.
Clark M G, Clerk L H, Newman J M B & Rattigan S. Interaction between metabolism and flow in tendon and muscle. Scand. J. Med. Sci. Sports, 10: 338-345, 2000
Clark M G, Barrett E J, Wallis M G, Vincent M A & Rattigan S. The microvasculature in insulin resistance and Type 2 diabetes. Seminars in Vascualr Medicine. 2: 21-31, 2002.
Rattigan S, Dora K A, Colquhoun E Q & Clark M G. Serotonin-mediated acute insulin resistance in the perfused rat hindlimb but not in incubated muscle: a role for the vascular system. Life Sci. 53: 1545-1555,1993.
Rattigan S, Appleby G J, Miller K A, Steen J T, Dora K A, Colquhoun E Q & Clark M G. Serotonin inhibition of 1-methylxanthine metabolism parallels its vasoconstrictor activity and inhibition of oxygen uptake in perfused rat hindlimb. Acta Physiol. Scand. 161: 161-169,1997.
Jarasch E D, Bruder G, Heid H W: Significance of xanthine oxidase in capillary endothelial cells. Acta Physiol. Scand. Suppl. 548:39-46,1986
Youd J M, Newman J M B, Clark M G, Appleby G J, Rattigan S, Tong A C Y & Vincent M A. Increased metabolism of infused 1-methylxanthine by working muscle. Acta Physiol. Scand. 166: 301-308,1999.
Rattigan S, Clark M G & Barrett E J. Haemodynamic actions of insulin in rat skeletal muscle: evidence for capillary recruitment. Diabetes, 46: 1381-1388, 1997.
Rattigan S, Clark M G & Barrett E J. Acute vasoconstriction-induced insulin resistance in vivo in rat muscle. Diabetes, 48: 564-569, 1999.
Vincent M A, Dawson D, Clark A D H, Lindner J, Rattigan S, Clark M G, Kaul S, Barrett E J. Physiologic hyperinsulinemia rapidly recruits capillaries in rat skeletal muscle in vivo. Diabetes submitted, 2000
Coggins M P, Fasy E, Lindner J, Jahn L, Kaul S, Barrett E J. Physiologic hyperinsulinemia increases skeletal muscle microvascular blood volume in healthy humans (Abstract). Diabetes 48: supplement: A220, 1999.
Barrett E, Fryburg D, Louard R, Gelfand R, McNulty P, Young L. Use of regional catherization methods to study the regulation of muscle protein metabolism. In: Nair K, editor. Protein metabolism in diabetes mellitus. Great Britain: Smith-Gordon; 1992. p 91-100.
Fryburg D A, Jahn L A, Hill S A, Oliveras D M, Barrett E J. Insulin and insulin-like growth factor-I enhance human skeletal muscle protein anabolism during hyperaminoacidemia by different mechanisms. Journal of Clinical Investigation 96:1722-1729,1995.
Louard R J, Fryburg D A, Gelfand R A, Barrett E J. Insulin sensitivity of protein and glucose metabolism in human forearm skeletal muscle. Journal of Clinical Investigation 90:2348-2354,1992.
Jayaweera A R, Kaul S. Quantifying myocardial blood flow with contrast echocardiography. American Journal of Cardiac Imaging 7(4): 317-35,1993.
Wei K, Jayaweera A R, Firoozan S, Linka A, Skyba D M, Kaul S. Quantification of myocardial blood flow with ultrasound-induced destruction of microbubbles administered as a constant venous infusion. Circulation 97(5): 473-83,1998
Wei K, Skyba D M, Firschke C, Jayaweera A R, Lindner J R, Kaul S. Interactions between microbubbles and ultrasound: in vitro and in vivo observations. Journal of the American College of Cardiology 29(5): 1081-8,1997.
Wei K, Kaul S. Recent advances in myocardial contrast echocardiography. Current Opinion in Cardiology 12(6): 539-46,1997
Capaldo B, Gastaldelli A, Antoniello S, Auletta M, Pardo F, Ciociaro D, Guida R, Ferrannini E, Sacca L. (1999) Splanchnic and leg substrate exchange after ingestion of a natural mixed meal in humans. Diabetes 48: 958-966.

1. A method of measuring changes in microvascular capillary flow in a predetermined region in a subject comprising:
(a) administering an ultrasound contrast medium to said subject such that said contrast medium reaches the microvascular capillaries in said predetermined region;
(b) measuring microvascular capillary blood flow volume and/or microvascular flow index of said capillaries;
(c) applying a defined signal to said subject; and,
(d) measuring changes in said microvascular capillary flow.

2. A method according to claim 1 wherein the measurement is made by ultrasound imaging.

3. A method according to claim 1 wherein said contrast medium is microbubbles.

4. A method according to claim 3 wherein said microbubbles are selected from albumin and/or phospholipid microbubbles.

5. A method according to claim 2 wherein said ultrasound image may be obtained by using contrast enhanced ultrasound.

6. A method according to claim 1 wherein said defined signal includes any signal potentially or actually capable of affecting microvascular capillary flow.

7. A method according to claim 6 wherein said defined signal includes any one or a combination of:
applying insulin to the capillary region of a subject;
applying a drug intended to increase or decrease microvascular capillary flow of a subject; and
causing a defined level of exercise to be applied to a muscle area associated with the capillaries of said subject.

8. A diagnostic method for differentiating normal from abnormal responses in microvascular capillaries to the application of a defined signal, said method comprising the use of the method of claim 1 in the following steps:
(a) measuring microvascular capillary flow in a predetermined region in a subject in response to said defined signal;
(b) measuring microvascular capillary flow in an analogous predetermined region in a control subject;
(c) comparing said flow rates.

9. A method of diagnosis characterised by the direct measurement of any change in microvascular capillary flow at the source in a subject using the methods of claim 1.

10. A method for determining changes in nutritive capillary recruitment in a subject comprising the use of the method of claim 1 in the following steps:
(a) measuring microvascular capillary flow in a subject in a first situation;
(b) measuring microvascular capillary flow in a subject in a second situation;
(c) comparing said flow rate.

11. A method of diagnosis of any one or a combination of conditions including Type I or Type II diabetes, hypertension, obesity and patients in critical care comprising the following steps:
(a) administering an ultrasound contrast medium to a subject in need of diagnosis such that said contrast medium reaches the microcapillaries of said subject;
(b) measuring microvascular capillary blood flow volume and br microvascular flow velocity index of said capillaries;
(c) applying a defined signal to said subject;
(d) measuring changes to said microvascular capillary flow wherein said measurement is made by ultrasound imaging;
(e) repeating steps (a) to (d) in a control subject and/or comparing know control result, such that any variation of results from the control indicates the existance of predisposition of said subject to said condition.

12. A method according to claim 11 wherein said defining signal is insulin.

13. A method according to claim 1 substantially as herein
before described with reference to the examples.
